(19) United States
(12) Patent Application Publication
(10) Pub. No.: US 2005/0112204 A1
(43) Pub. Date: May 26, 2005

(54) Pharmaceutical formulations

(75) Inventors: Michael John Humphrey, Sandwich (GB); Tobin Andrew Payne-Cook, Sandwich (GB)

Correspondence Address: AGOURON PHARMACEUTICALS, INC., 10350 NORTH TORREY PINES ROAD, LA JOLLA, CA, 92037, US

(73) Assignee: Pfizer Inc.

(21) Appl. No.: 10/997,022

(22) Filed: Nov. 23, 2004

Related U.S. Application Data
(60) Provisional application No. 60/536,412, filed on Jan. 14, 2004.

(30) Foreign Application Priority Data
Nov. 25, 2003 (GB) 0327390.1

(51) Int. Cl. A61K 31/506; A61K 9/14

(52) U.S. Cl. 424/486; 514/256

(57) ABSTRACT

This invention relates to a new pharmaceutical formulation of voriconazole, in particular a formulation comprising one or more poloxamers.

This application claims priority to United Kingdom Application Number 0327390.1, filed Nov.25, 2003, and U.S. Provisional Application No. 60/536,412, filed Jan. 14, 2004, which are hereby incorporated by reference.

This invention relates to a new pharmaceutical formulation of voriconazole, in particular a formulation comprising one or more poloxamers.

### BACKGROUND OF THE INVENTION

(2R,3S)-2-(2,4-Difluorophenyl)-3-(5-fluoro-4-pyrimidinyl)-1-(1H-1,2,4-triazol-1-yl)-butan-2-ol, also known as voriconazole, is disclosed in EPA440372 (incorporated in its entirety herein by reference); see in particular Example 7. Voriconazole has the following structure: 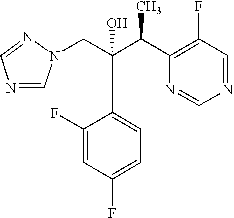
and is useful in the treatment of fungal infections. Voriconazole has a low aqueous solubility (0.61 mg/ml @ pH 7), and is not stable in water (an inactive enantiomer is formed from recombination of the retro-aldol products of hydrolysis). Thus, development of an aqueous intravenous formulation with a sufficient shelf life is difficult. These problems are magnified by the semi-polar nature of the compound (log D=1.8) which means that it is not generally solubilised by conventional means such as oils, surfactants or water miscible co-solvents.

Accordingly there is a need for a formulation of voriconazole that increases its solubility and aqueous stability.

WO98/58677 discloses that the solubility of voriconazole in water can be increased by molecular encapsulation with sulphoalkylether cyclodextrin derivatives of the type disclosed in WO91/11172, particularly β-cyclodextrin derivatives wherein the cyclodextrin ring is substituted by sulphobutyl groups. There are complex manufacturing issues associated with cyclodextrin formulations that significantly increase cost of goods.

WO97/28169 (see Examples 3-5) discloses a phosphate pro-drug of voriconazole, which exhibits increased solubility and aqueous stability. However, the pro-drug does not exhibit 100% bioequivalence to voriconazole, such that cost of goods is again significantly increased.

Poloxamers in general are non-toxic polymeric non-ionic surfactants comprising ethylene oxide (EO) and propylene oxide (PO) monomers arranged as ABA block copolymers: (EO)ₓ-(PO)_{y}-(EO)ₓ, where x and y can be varied to alter the hydrophilic-lipophilic balance. The ethoxylated regions are hydrophilic, while the propoxylated region is hydrophobic. Above certain concentrations in water the surfactants tend to form micelles. These agglomerates of surfactant molecules present their hydrophilic portions to water and internalise the hydrophobic portions. Conversely, each surfactant has a minimum concentration in water below which micelles disperse, the critical micelle concentration (cmc). These micelles can readily interact with other substances and if the substance is sufficiently hydrophobic the substance can be entirely internalised or enveloped within the micelle, or otherwise form an association, thereby effectively solubilising the substance in water.

The pharmaceutical acceptability of various poloxamers is well established, with certain species approved for parenteral administration.

However, there have been problems with targeting and dispensing drugs using poloxamers. Munshi, et al., [Cancer Letters, 118 (1997), 13-19] found that it was not possible for the drug to act in a normal manner, unless ultrasound was used to disrupt the micelles. The requirement of the use of ultrasound is expensive and undesirable.

Kabanov, et al., [Journal of Controlled Release, 22 (1992), 141-158] disclose a self-assembling supramolecular complex comprising drug, poloxamer and antibodies to try to target the drug contained within the complex. Targeting the micelles by incorporating antibodies is not practical for a more general application.

WO01/64187 discloses an aqueous, micellar poloxamer preparation comprising Propofol wherein Propofol is solubilised by a mix of two poloxamers. The stability of the resulting mixed micellar preparation is dependent upon the presence of Propofol. Typically in a mixed micelle, with poloxamers of differing PO length, when the PO blocks of different poloxamers align, either a hole is left in the micellar interior, or part of the EO block of the shorter poloxamer must align with the PO of the larger molecule. This is not generally thermodynamically stable and with poloxamers that are substantially different it happens virtually not at all.

It has now, surprisingly, been found that micellar preparations of poloxamers containing voriconazole significantly improve the aqueous solubility of voriconazole, are stable, and that such preparations are effective administrative forms of voriconazole.

Accordingly the invention provides an aqueous micellar poloxamer preparation comprising voriconazole.

### SUMMARY OF THE INVENTION

In one embodiment the invention provides an aqueous micellar poloxamer preparation comprising voriconazole and a single poloxamer.

In another embodiment the invention provides an aqueous micellar poloxamer preparation comprising voriconazole and a combination of two or more poloxamers.

### BRIEF DESCRIPTION OF THE FIGURES

FIG. 1 shows the solubility of voriconazole in water and the poloxamer solutions (see Example 2).

### DETAILED DESCRIPTION OF THE INVENTION

The formulations of the invention include voriconazole and pharmaceutically acceptable salts, solvates, or derivatives thereof (wherein derivatives include complexes, prodrugs and isotopically-labelled compounds, as well as salts and solvates thereof).

Pharmaceutically acceptable salts of voriconazole include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of voriconazole may be readily prepared by mixing together solutions of voriconazole and the desired acid or base, as appropriate. The salt may precipitate from solution and be collected by filtration or may be recovered by evaporation of the solvent. The degree of ionisation in the salt may vary from completely ionised to almost non-ionised.

Voriconazole or a pharmaceutically acceptable salt or derivative thereof may exist in both unsolvated and solvated forms. The term ‘solvate’ is used herein to describe a molecular complex comprising voriconazole, or a pharmaceutically acceptable salt or derivative thereof, and one or more pharmaceutically acceptable solvent molecules, for example, ethanol. The term ‘hydrate’ is employed when said solvent is water.

All references herein to voriconazole include references to salts thereof, and to solvates of voriconazole or salts thereof.

The formulations of the invention include voriconazole as hereinbefore defined, polymorphs and prodrugs thereof and isotopically-labelled voriconazole or a pharmaceutically acceptable salt or solvate thereof.

As stated, the invention includes all polymorphs and so-called ‘prodrugs’ of voriconazole. In respect of the latter, certain derivatives of voriconazole which may have little or no pharmacological activity themselves can, when administered into or onto the body, be converted into voriconazole, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs in accordance with the invention can, for example, be produced by replacing appropriate functionalities present in voriconazole with certain moieties known to those skilled in the art as ‘pro-moieties’ as described, for example, in “Design of Prodrugs” by H Bundgaard (Elsevier, 1985).

Voriconazole contains an alcohol functionality (—OH), therefore some examples of prodrugs in accordance with the invention may include an ester or ether thereof, for example, by replacement of the hydrogen by phosphorylation to provide (2R,3S)-2-(2,4-Difluorophenyl)-3-(5-fluoro-4-pyrimidinyl)-1-(1H-1,2,4-triazol-1-yl)-2-butyl dihydrogen phosphate as described in WO97/28169 (incorporated in its entirety herein by reference); see in particular Example 3. Further examples of replacement groups in accordance with the foregoing examples and examples of other prodrug types in accordance with the invention may be found in the aforementioned references.

The present invention also includes isotopically-labelled voriconazole or a pharmaceutically acceptable salt, solvate or derivative thereof, wherein one or more atoms are replaced by atoms having the same atomic number, but an atomic mass or mass number different from the atomic mass or mass number usually found in nature.

Examples of isotopes suitable for inclusion in isotopically-labelled voriconazole or a pharmaceutically acceptable salt, solvate or derivative thereof, include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³¹P and ³²P, and sulphur, such as ³⁵S.

Isotopically-labelled voriconazole or a pharmaceutically acceptable salt, solvate or derivative thereof, for example, incorporating a radioactive isotope, may be useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled voriconazole or a pharmaceutically acceptable salt, solvate or derivative thereof, can generally be prepared by conventional techniques known to those skilled in the art or by processes analogous to those described in EPA440372 or WO97/06160 (incorporated in its entirety herein by reference) using an appropriate isotopically-labelled reagent in place of the non-labelled reagent previously employed.

Pharmaceutically acceptable solvates in accordance with the invention include those wherein the solvent of crystallization may be isotopically substituted, e.g. D₂O, d₆-acetone, d₆-DMSO.

It is a particular advantage of the invention that small amounts of pharmaceutically acceptable compounds can be used to solubilise voriconazole. In contrast to WO01/64187 it has, surprisingly, been found that a simple aqueous formulation comprising voriconazole, a poloxamer and water is sufficient to manufacture a stable and efficacious medicament for the treatment of fungal infections.

Poloxamers are generally unreactive and non-responsive to any other additives. Therefore addition of further suitable substances to the formulation for the purpose of parenteral administration is typically tolerated.

Preparation of formulations of the present invention is generally straightforward. Although the constituents can be added in any sequence, as voriconazole is poorly soluble in water, the commercially desirable method of manufacture is to prepare a poloxamer solution followed by the addition of voriconazole. Heating of the water and poloxamer can generally increase the speed of micelle formation.

Poloxamers for use according to the invention, comprising EO and PO monomers arranged as ABA block copolymers (EO)ₓ-(PO)_{y}-(EO)ₓ, typically have x values ranging from about 85 to 150, such as about 90 to 135, for example about 95 to 120; and have y values ranging from about 40 to 85, such as about 44 to 75, for example about 56 to 70.

Poloxamers for use according to the invention typically have an average molecular weight of from about 9800 to 17400, such as about 9800 to 14600, for example about 12000 to 13000.

Poloxamer concentrations of about 15% are typically useful in the invention, but concentrations of poloxamers can be selected by those skilled in the art and will generally range from about 5 to 30%, such as about 10 to 20%, for example about 12 to 18%. Preferred poloxamer concentrations are those that do not gel at body temperature.

In a further aspect the invention provides a method of treating a fungal infection in a mammal, including man, which comprises administration to said mammal an effective amount of an aqueous micellar poloxamer preparation comprising voriconazole.

In a further aspect the invention provides the use of an aqueous micellar poloxamer preparation comprising voriconazole in the manufacture of a medicament for treating a fungal infection in a mammal, including man.

In a further aspect the invention provides an aqueous micellar poloxamer preparation comprising voriconazole for use in treating a fungal infection in a mammal, including man.

Fungal infections which may be treated by voriconazole have been extensively described in the literature, including EPA440372, and include topical infections caused by, inter alia, Candida spp, Trichophyton spp, Microsporum spp or Epidermophyton floccosum; mucosal infections caused by Candida spp; systemic infections caused by, inter alia, Candida spp, Cryptococcus neoformans, Aspergillus spp, Fusarium spp, Scedosporium spp, Coccidioides immitis, Paracoccidioides brasiliensis, Histoplasma spp or Blastomyces dermatiditis. Particular infections include, but are not limited to, fungal peritonitis, vaginal candidiasis and allergic rhinosinusistis. It will be appreciated that reference to treatment is intended to include prophylaxis as well as the alleviation of established symptoms.

Likewise, EPA440372 also describes a suitable dose for the parenteral administration of voriconazole. A typical dose is between 0.1 to 25 mg/kg, such as 0.5 to 20 mg/kg, for example 1 to 10 mg/kg, administered twice a day. It will be appreciated that the precise therapeutic dose of voriconazole will depend on the age and condition of the patient and the nature of the condition to be treated and will be at the ultimate discretion of the attendant physician.

Formulations of the present invention suitable for parenteral administration need very few constituents, but it is generally preferred that an injectable solution is made up with saline to provide a solution which is iso-osmotic with blood. These formulations can be sterilised after preparation. They can be provided in any suitable form and in any suitable containers appropriate to maintaining sterility. It is generally preferred that formulations of the present invention are provided in a form suitable for direct injection. It will be readily appreciated by those skilled in the art how to administer formulations of the present invention to a human or animal.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

The formulations of the invention may also be administered topically to the skin or mucosa, that is, dermally or transdermally. Typical formulations for this purpose include gels, hydrogels, lotions, solutions, creams, ointments, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The formulations of the invention can also be administered intranasally or by inhalation, typically as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane.

The pressurised container, pump, spray, atomizer, or nebuliser contains a solution of the formulations of the invention comprising, for example, ethanol (optionally, aqueous ethanol) or a suitable alternative agent for dispersing, solubilising, or extending release of the active, the propellant(s) as solvent and an optional surfactant, such as sorbitan trioleate, oleic acid, or an oligolactic acid.

A suitable solution formulation for use in an atomiser using electrohydrodynamics to produce a fine mist may contain from 1 μg to 20 mg of voriconazole or pharmaceutically acceptable salts, solvates, or derivatives thereof per actuation and the actuation volume may vary from 1 μl to 100 μl. A typical formulation may comprise a formulation of the invention, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of aerosols, the dosage unit is determined by means of a valve which delivers a metered amount. Units in accordance with the invention are typically arranged to administer a metered dose or “puff” containing from 1 μg to 10 mg of voriconazole or pharmaceutically acceptable salts, solvates, or derivatives thereof. The overall daily dose will typically be in the range 1 μg to 200 mg which may be administered in a single dose or, more usually, as divided doses throughout the day.

The formulations of the invention may also be administered directly to the eye or ear, typically in the form of drops of solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

### EXAMPLE 1

### Preparation of an I.V. Formulation of Voriconazole

| Ingredient | mg/ml | |
|---|---|---|
| Voriconazole, Pfizer | 3.000 | |
| Poloxamer F127, BASF | 150.000 | |
| Water for injections, Ph. Eur | to 1.000 | ml |
| Total | 1.000 | ml |

Method:
1. With constant stirring, add the F127 to 80% of the final volume of water for injections and continue to stir until all the F127 has dissolved.
2. Add the voriconazole and dissolve with stirring.
3. Make the solution up to volume with water for injections.
4. Filter the resulting solution through a sterile 0.2 mm nylon filter into a sterile container.

Poloxamer F127, BASF, has a molecular weight of 12600, an x value of 100 and a y value of 65.

### EXAMPLE 2

### Stability of an I.V. Formulation of Voriconazole

Voriconazole aqueous solutions of 0.5 and 1 mg/ml were prepared in water (20 ml). Corresponding aqueous solutions of 2, 3 and 4 mg/ml were prepared in aqueous poloxamer solutions (20 ml), as follows:

| F127 (% w/w) | Comments on viscosity |
|---|---|
| 15 | Viscous liquid |
| 10 | Fluid liquid |

The samples were prepared in 50 ml glass vials, and sonicated in an ultrasonic bath for 30 minutes. The solutions were allowed to cool, then visually assessed immediately and after 18 hours.

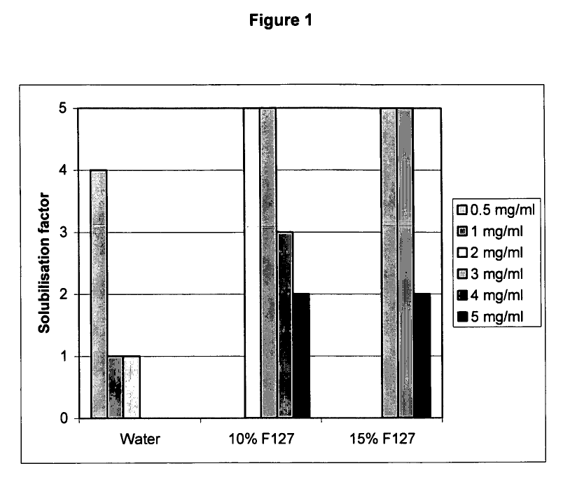

The solubility of voriconazole in water and the poloxamer solutions are shown in FIG. 1. The solubilization factor on the y-axis is a code for the level of solubility visually observed. The numbers are explained in the table below:

| “Solubilization factor” | Definition |
|---|---|
| 1 | Not Dissolved |
| 2 | A lot of settled particulates present |
| 3 | Some particulates present |
| 4 | Trace of particulates observed |
| 5 | Completely Dissolved |

The visual solubility of voriconazole in water complements the previously measured data (0.61 mg/ml at 22° C., pH 7). The graph shows virtually complete solubility at 0.5 mg/ml under ambient conditions, while it is clearly insoluble at 1 mg/ml.

The results show that voriconazole is soluble up to 2 mg/ml in 10% F127 solution, which represents a three to four fold increase in solubility in comparison with water alone, and is soluble up to 3 mg/ml in 15% F127 solution, representing a five to six fold increase in solubility.

1. An aqueous micellar poloxamer formulation comprising voriconazole and one or more poloxamer.

2. A formulation according to claim 1 comprising voriconazole and a single poloxamer.

3. A formulation according to claim 1 comprising voriconazole and a combination of two or more poloxamers.

4. A formulation according to claim 1 comprising a poloxamer having x values in a ranging from 85 to 150 and y values ranging from 40 to 85.

5. A formulation according to claim 1 comprising a poloxamer having an average molecular weight in a range from 9800 to 17400.

6. A formulation according to claim 1, wherein the poloxamer concentration is in a range from 5% to 30%.

7. A method of treating a fungal infection in a mammal, including man, which comprises administration to said mammal an effective amount of a formulation according to claim 1.

8. The use of a formulation according to claim 1 in the manufacture of a medicament for treating a fungal infection in a mammal, including man.

9. A formulation according claim 1 for use in treating a fungal infection in a mammal, including man.
